(19) United States
(12) Patent Application Publication
(10) Pub. No.: US 2007/0162061 A1
(43) Pub. Date: Jul. 12, 2007

(54) TISSUE EXCISION DEVICES AND METHODS

(75) Inventors: Bryce Way, San Jose, CA (US); Donald Schomer, Englewood, CO (US); Minh Tran, Westminster, CA (US); Murray David Solsberg, Englewood, CO (US)

Correspondence Address: CONLEY ROSE, P.C., P. O. BOX 3267, HOUSTON, TX, 77253-3267, US

(73) Assignee: X-STEN, CORP., San Jose, CA (US)

(21) Appl. No.: 11/556,213

(22) Filed: Nov. 3, 2006

Related U.S. Application Data
(60) Provisional application No. 60/733,552, filed on Nov. 4, 2005.

(51) Int. Cl. A61B 17/32

(52) U.S. Cl. 606/167

(57) ABSTRACT

A device for percutaneously excising tissue. In an embodiment, the device comprises an elongate body including a first member having a distal cutting end and a second member that slidingly engages the first member. In addition, the second member includes a tissue capture chamber having an opening facing the first member. Further, the first member is moveable relative to the second member between an opened position and a closed position, wherein the first member is disposed across the tissue capture chamber of the second member when the first member is in the closed position.

### CROSS-REFERENCE TO RELATED APPLICATIONS

This application claims benefit of U.S. provisional application Ser. No. 60/733,552 filed Nov. 4, 2005, and entitled “Contoured Tissue Retraction Device,” which is hereby incorporated herein by reference in its entirety.

### STATEMENT REGARDING FEDERALLY SPONSORED RESEARCH OR DEVELOPMENT

Not Applicable.

### BACKGROUND

1. Field of the Invention

The present invention relates generally to minimally invasive methods, devices and systems for treating spinal disorders using imaging guidance. More particularly, the present invention relates to devices and methods to reduce stenosis and increase the cross-sectional area of the spinal canal available for the spinal cord. Still more particularly, the present invention relates to devices and methods to percutaneously excise portions of an enlarged ligamentum flavum.

2. Background Information

The vertebral column (spine, spinal column, backbone) forms the main part of the axial skeleton, provides a strong yet flexible support for the head and body, and protects the spinal cord disposed in the vertebral canal, which is formed within the vertebral column. The vertebral column comprises a stack of vertebrae with an intervertebral disc between adjacent vertebra. The vertebrae are stabilized by muscles and ligaments that hold the vertebrae in place and limit the movements of the individual vertebra.

As illustrated in FIG. 1, each vertebra 10 includes a vertebral body 12 that supports a vertebral arch 14. A median plane 210 generally divides each vertebra 10 into two substantially equal lateral sides. Vertical body 12 has the general shape of a short cylinder and is anterior to the vertebral arch 14. The vertebral arch 14 together with vertebral body 12 encloses a space termed the vertebral foramen 15. The succession of vertebral foramen 15 in adjacent vertebra 10 along the vertebral column define the vertebral canal (spinal canal), which contains the spinal cord 28.

Vertebral arch 14 is formed by two pedicles 24 which project posteriorly to meet two laminae 16. The two laminae 16 meet posteriomedially to form the spinous process 18. At the junction of pedicles 24 and laminae 16, six processes arise. Two transverse processes 20 project posterolaterally, two superior articular processes 22 project generally superiorly and are positioned superior to two inferior articular processes 25 that generally project inferiorly.

The vertebral foramen 15 is generally an oval shaped space that contains and protects the spinal cord 28. Spinal cord 28 comprises a plurality of nerves 34 surrounded by cerebrospinal fluid (CSF) and an outermost sheath/membrane called the dural sac 32. The CSF filled dural sac 32 containing nerves 34 is relatively compressible. Posterior to the spinal cord 28 within vertebral foramen 15 is the ligamentum flavum 26. Laminae 16 of adjacent vertebral arches 14 in the vertebral column are joined by the relatively broad, elastic ligamentum flavum 26.

In degenerative conditions of the spine, narrowing of the spinal canal (stenosis) can occur. Lumbar spinal stenosis is often defined as a dural sac cross-sectional area less than 100 mm² or an anterior-posterior (AP) dimension of the canal of less than 10-12 mm for an average male.

The source of many cases of lumbar spinal stenosis is thickening of the ligamentum flavum (e.g., ligamentum flavum 26). Spinal stenosis may also be caused by subluxation, facet joint hypertrophy, osteophyte formation, underdevelopment of spinal canal, spondylosis deformans, degenerative intervertebral discs, degenerative spondylolisthesis, degenerative arthritis, ossification of the vertebral accessory ligaments and the like. A less common cause of spinal stenosis, which usually affects patients with morbid obesity or patients on oral corticosteroids, is excess fat in the epidural space. The excessive epidural fat compresses the dural sac, nerve roots and blood vessels contained therein, and results in back, leg pain and weakness and numbness of the legs. Spinal stenosis may also affect the cervical and, less commonly, the thoracic spine.

Patients suffering from spinal stenosis are typically first treated with exercise therapy, analgesics, and anti-inflammatory medications. These conservative treatment options frequently fail. If symptoms are severe, surgery is required to decompress the spinal cord and nerve roots.

In some conventional surgical procedures to correct stenosis in the lumbar region, an incision is made in the back, and the muscles and supporting structures are stripped away from the spine, exposing the posterior aspect of the vertebral column. The thickened ligamentum flavum is then exposed by removal of a portion of the vertebral arch (e.g., vertebral arch 14), often at the laminae (e.g., laminae 16), covering the back of the spinal canal (laminectomy). The thickened ligamentum flavum ligament can then be excised by sharp dissection with a scalpel or punching instruments, such as a Kerison punch that is used to remove small chips of tissue. The procedure is performed under general anesthesia. Patients are usually admitted to the hospital for approximately five to seven days depending on the age and overall condition of the patient. Patients usually require between six weeks and three months to recover from the procedure. Further, many patients need extended therapy at a rehabilitation facility to regain enough mobility to live independently.

Much of the pain and disability after an open laminectomy results from the tearing and cutting of the back muscles, blood vessels, supporting ligaments, and nerves that occurs during the exposure of the spinal column. Also, because the spine-stabilizing back muscles and ligaments are stripped and detached from the spine during the laminectomy, these patients frequently develop spinal instability post-operatively.

Less invasive techniques offer the potential for reduced post-operative pain and faster recovery compared to traditional open surgery. Percutaneous interventional spinal procedures can be performed with local anesthesia, thereby sparing the patient the risks and recovery time required with general anesthesia. In addition, there is less damage to the paraspinal muscles and ligaments with minimally invasive techniques, thereby reducing pain and preserving these important stabilizing structures.

Various techniques for minimally invasive treatment of the spine are known. Microdiscectomy is performed by making a small incision in the skin and deep tissues to create a portal to the spine. A microscope is then used to aid in the dissection of the adjacent structures prior to discectomy. The recovery for this procedure is much shorter than traditional open discectomies. Percutaneous discectomy devices with fluoroscopic guidance have been used successfully to treat disorders of the disc but not to treat spinal stenosis or the ligamentum flavum directly. Arthroscopy or direct visualization of the spinal structures using a catheter or optical system have also been proposed to treat disorders of the spine including spinal stenosis, however these devices still use miniaturized standard surgical instruments and direct visualization of the spine similar to open surgical procedures. These devices and techniques are limited by the small size of the canal and these operations are difficult to perform and master. In addition, these procedures are painful and often require general anesthesia. Further, the arthroscopy procedures are time consuming and the fiber optic systems are expensive to purchase and maintain.

Hence, it remains desirable to provide simple methods, techniques, and devices for treating spinal stenosis and other spinal disorders without requiring open surgery. It is further desired to provide a system whereby the risk of damage to the dural sac containing the spinal nerves may be reduced.

### SUMMARY OF THE PREFERRED EMBODIMENTS

In accordance with at least one embodiment of the invention, a device for percutaneously excising tissue comprises an elongate body including a first member having a distal cutting end and a second member that slidingly engages the first member. In addition, the second member includes a tissue capture chamber having an opening facing the first member. Further, the first member is moveable relative to the second member between an opened position and a closed position, wherein the first member is disposed across the tissue capture chamber of the second member when the first member is in the closed position.

In accordance with another embodiment of the invention, a method for treating stenosis in a spine of a patient comprises providing a tissue excision device. In addition, the method comprises positioning the tissue excision device adjacent the region of interest. Further, the method comprises opening the cavity of the tissue excision device by sliding the first member relative to the second member. Still further, the method comprises inserting the tissue excision device into tissue in the region of interest. Moreover, the method comprises closing the cavity of the tissue excision device by sliding the first member relative to the second member. In addition, the method comprises capturing an excised tissue segment within the cavity of the second member.

In accordance with another embodiment of the invention, a kit for performing a procedure on a spine comprises a tissue excision device. The tissue excision device comprises a first moveable member and a second moveable member including a tissue capture chamber having an opening facing the first moveable member, wherein the second moveable member slidingly engages the first moveable member. In addition, the kit comprises a tissue retrieval device.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

### BRIEF DESCRIPTION OF THE DRAWINGS

For a more complete understanding of the disclosure, reference is made to the accompanying drawings, wherein:

FIG. 1 is cross-section of the spine viewed from the space between two vertebrae, showing the upper surface of one vertebra and the spinal canal with the dural sac and a normal (un-stenosed) ligamentum flavum therein;

FIG. 2 is cross-section of the spine viewed from the space between two vertebrae, showing the upper surface of one vertebra and the spinal canal with the dural sac and a thickened (stenosed) ligamentum flavum therein;

FIG. 3 is an enlarged cross-section of a vertebral foramen, showing a safety zone created by compression of the dural sac;

FIG. 4 is the cross-section of FIG. 3, showing a tissue excision device positioned in the ligamentum flavum according to an ILAMP procedure;

FIG. 5 is the cross-section of FIG. 3, showing a tissue excision tool positioned in the ligamentum flavum according to an alternative MILD procedure;

FIG. 6 is a partial cross-section of the lumbar portion of the vertebral column taken along lines 6-6 of FIG. 1;

FIG. 7 is the cross-section of FIG. 6, showing the orientation of an imaging tool relative to the vertebral column;

FIG. 8 is the cross-section of FIG. 6, showing the orientation of a tissue excision device relative to the vertebral column;

FIG. 9 is a side view of the distal portion of an embodiment of a tissue excision device in the closed position;

FIG. 10 is a cross-sectional view of the tissue excision device of FIG. 9;

FIG. 11 is a cross-sectional view of the tissue excision device of FIG. 9 taken along line 11-11;

FIG. 12 is a side view of the tissue excision device of FIG. 9 in the opened position;

FIG. 13 is a side view of the distal portion of another embodiment of a tissue excision device in the opened position;

FIG. 14 is a side view of the tissue excision device of FIG. 13 in the closed position;

FIG. 15 is a cross-sectional view of the tissue excision device of FIG. 14 taken along line 12-12;

FIGS. 16 to 18 are selected sequential side views of the tissue excision device of FIG. 9 penetrating tissue and excising a segment of the tissue;

FIGS. 19 and 20 are selected cross-sectional views of another embodiment of a tissue excision device including an ejector employed to empty the tissue excision device;

FIGS. 21 and 22 are selected cross-sectional view of another embodiment of a tissue excision device including a tissue retrieval device employed to retrieve and remove an excised tissue segment from the tissue excision device;

FIG. 23 is an enlarged view of the distal end of the tissue retrieval device of FIGS. 18 and 19; and

FIG. 24 is a view of an embodiment of a tissue excision device having a contoured body inserted into the ligamentum flavum.

### DETAILED DESCRIPTION OF THE PREFERRED EMBODIMENTS

The following discussion is directed to various embodiments of the invention. Although one or more of these embodiments may be presently preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

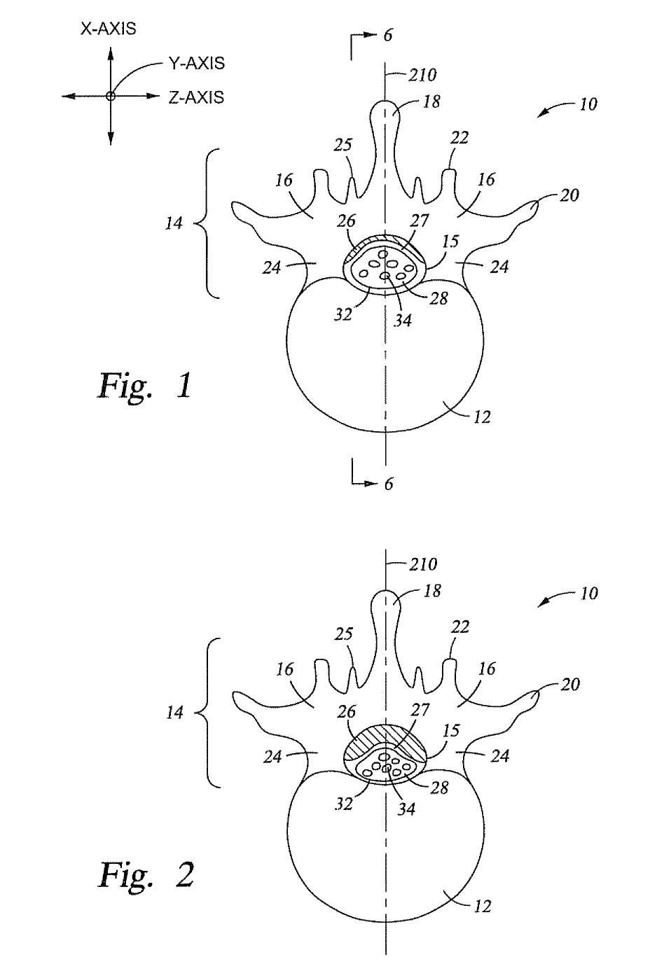

For purposes of this discussion, the x-, y-, and z-axes are shown in FIGS. 1, 3, 6, 7, and 8 to aid in understanding the descriptions that follow. The x-, y-, and z-axes have been assigned as follows. The x-axis is perpendicular to the longitudinal axis of the vertebral column and perpendicular to the coronal/frontal plane (i.e., x-axis defines anterior vs. posterior relationships). The y-axis runs substantially parallel to the vertebral column and perpendicular to the transverse plane (i.e., y-axis defines superior vs. inferior relationships). The z-axis is perpendicular to the longitudinal axis of the vertebral column and perpendicular to the median/midsagittal plane (i.e., z-axis defines the lateral right and left sides of body parts). The set of coordinate axes (x-, y-, and z-axes) are consistently maintained throughout although different views of vertebrae and the spinal column may be presented.

It is to be understood that the median/midsagittal plane passes from the top to the bottom of the body and separates the left and the right sides of the body, and the spine, into substantially equal halves (e.g., two substantially equal lateral sides). Further, it is to be understood that the frontal/coronal plane essentially separates the body into the forward (anterior) half and the back (posterior) half, and is perpendicular to the median plane. Still further, it is to be understood that the transverse plane is perpendicular to both the median plane and coronal plane and is the plane which divides the body into an upper and a lower half.

The Spinal Canal and Spinal Stenosis

Referring again to FIG. 1, vertebral foramen 15 contains a portion of the ligamentum flavum 26, spinal cord 28, and an epidural space 27 between ligamentum flavum 26 and spinal cord 28. Spinal cord 28 comprises a plurality of nerves 34 surrounded by cerebrospinal fluid (CSF) contained within dural sac 32. Nerves 34 normally comprise only a small proportion of the dural sac 32 volume. Thus, CSF filled dural sac 32 is somewhat locally compressible, as localized pressure causes the CSF to flow to adjacent portions of the dural sac. Epidural space 27 is typically filled with blood vessels and fat. The posterior border of the normal epidural space 27 generally defined by the ligamentum flavum 26, which is shown in its normal, non-thickened state in FIG. 1.

FIG. 2 illustrates a case of spinal stenosis resulting from a thickened ligamentum flavum 26. Since vertebral foramen 15 is defined and surrounded by the relatively rigid bone its volume is essentially constant. Thus, thickening of ligamentum flavum 26 within vertebral foramen 15 can eventually result in compression of spinal cord 28. In particular, the thickened ligamentum flavum 26 may exert a compressive force on the posterior surface of dural sleeve 32. In addition, thickening of ligamentum flavum 26 may compress the blood vessels and fat occupying epidural space 27.

Compression of spinal cord 28, particularly in the lumbar region, may result in low back pain as well as pain or abnormal sensations in the legs. Further, compression of the blood vessels in the epidural space 27 that houses the nerves of the cauda equina may result in ischemic pain termed spinal claudication.

In order to relieve the symptoms associated with a thickened or enlarged ligamentum flavum 26, methods, techniques, and devices described herein may be employed to reduce the compressive forces exerted by the thickened ligamentum flavum on spinal cord 28 and the blood vessels in epidural space 27 (e.g., decompress spinal cord 28 and blood vessels in epidural space 27). In particular, compressive forces exerted by the thickened/enlarged ligamentum flavum 26 may be reduced by embodiments of a minimally invasive ligament decompression (MILD) procedure. In some embodiments, the MILD procedure may be performed percutaneously to reduce the size of ligamentum flavum 26 by excising portions of enlarged ligamentum flavum 26. In particular, in some embodiments of the MILD procedure, the ligamentum flavum 26 is accessed, cut and removed ipsilaterally (i.e., on the same side of vertebral arch 14) by a percutaneous caudal-cranial approach. Such an embodiment of the MILD procedure may be described hereinafter as Ipsilateral Approach MILD Procedure (ILAMP).

Creation of a Safety Zone

As shown in FIGS. 1 and 2, ligamentum flavum 26 is posteriorly apposed to spinal cord 28 within vertebral foramen 15. Thus, placement of tools within ligamentum flavum 26 to excise portions of ligamentum flavum 26 creates a risk of for inadvertent damage to the spinal cord 28, dural sac 32, and/or nerves 34. Thus, in preferred embodiments of the procedures described herein, prior to insertion of tissue excision devices into the ligamentum flavum 26, a gap is created between ligamentum flavum 26 and spinal cord 28 to provide a safety zone between ligamentum flavum 26 and spinal cord 28.

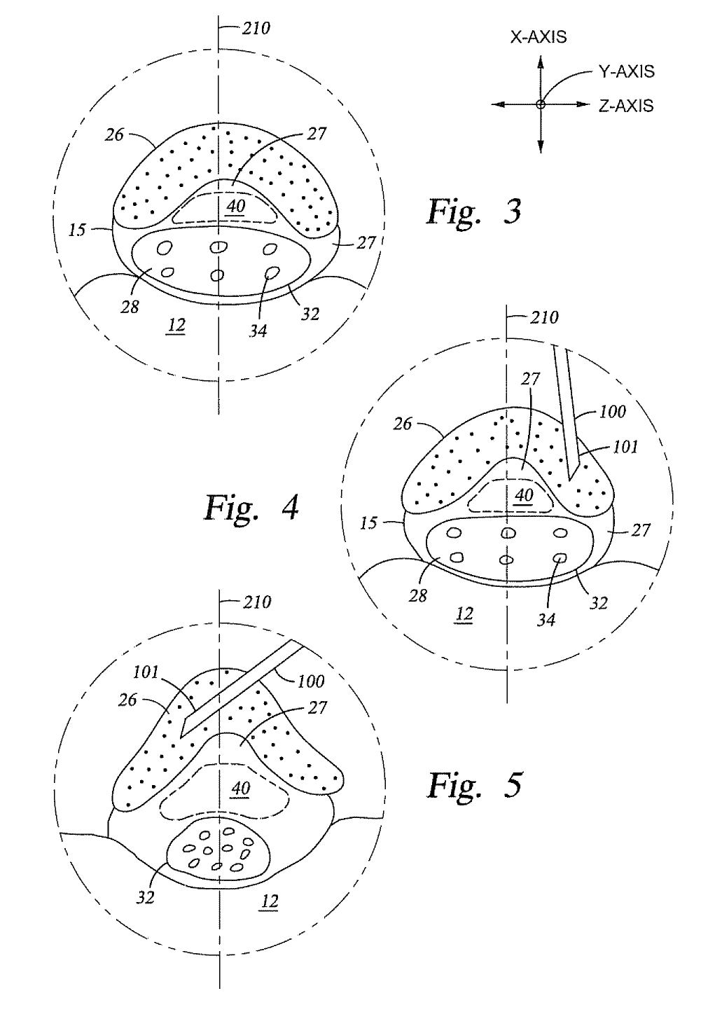

Referring now to FIG. 3, an enlarged cross-sectional view of a vertebral foramen 15 within a vertebra (e.g., vertebra 10) is illustrated. Vertebral foramen 15 includes epidural space 27 and spinal cord 28 containing nerves 34 and CSF within dural sac 32. Further, a thickened/enlarged ligamentum flavum 26 extends into vertebral foramen 15. To reduce the risk of damage to dural sac 32 and spinal cord 28, a safety zone 40 is created between ligamentum flavum 26 and dural sac 32 in the manner described below.

As previously described, spinal cord 28 comprises nerves 34 surrounded by CSF and is contained within dural sac 32. Since more than 90% of the volume of dural sac 32 in the lumbar region is filled by CSF, dural sac 32 is highly compressible. Thus, even when stenosis is causing compression of spinal cord 28, in most cases it is possible to temporarily compress spinal cord 28 further. Thus, according to preferred embodiments, dural sac 32 is further compressed in the region of interest by injecting a fluid or medium into epidural space 27 to create safety zone 40. The fluid may be injected into the epidural space 27 with an insertion member, such as a needle. The presence of the injected fluid comprising safety zone 40 gently applies an additional compressive force to the outer surface of dural sac 32 so that at least a portion of the CSF within dural sac 32 is forced out of dural sac 32 in the region of interest, resulting in safety zone 40 between dural sac 32 and ligamentum flavum 26.

According to some embodiments, dural sac 32 is compressed by injecting a standard radio-opaque non-ionic myelographic contrast medium or other imagable or non-imagable medium into epidural space 27 in the region of interest. This is preferably accomplished with a percutaneous injection. Sufficient injectable fluid is preferably injected to displace the CSF out of the region of interest and compress dural sac 32 to at least a desired degree. The injected medium is preferably substantially contained within the confines of epidural space 27 extending to the margins of the dural sac 32. The epidural space is substantially watertight and the fatty tissues and vascularization in epidural space 27, combined with the viscous properties of the preferred fluids, serve to substantially maintain the injected medium in the desired region of interest. This method for protecting spinal cord 28 column may be referred to hereinafter as “contrast-guided dural protection.”

Referring now to FIGS. 4 and 5, once safety zone 40 has been created, a tissue excision tool or device 100 may be inserted into ligamentum flavum 26. More specifically, the distal cutting end 101 of tissue excision device 100 is inserted into ligamentum flavum 26 in preparation for excising portions of enlarged ligamentum flavum 26. Tissue excision device 100 may comprise any suitable device, tool or instrument for decompressing an enlarged ligamentum flavum 26 and relieving spinal stenosis caused by the enlarged ligamentum flavum. A variety of suitable tissue excision devices, including distal cutting ends, are disclosed in U.S. application Ser. Nos. 11/193,581, 11/461,036, 60/733,754, 60/733,552, and 11/461,045, each of which is hereby incorporated herein by reference in its entirety.

Although tissue excision device 100 is shown as directly accessing ligamentum flavum 26 in FIGS. 4 and 5 (i.e., without guidance from a cannula or portal), it should be appreciated that tissue excision device 100 may alternatively percutaneously access ligamentum flavum 26 via a cannula or other portal device. For instance, in some embodiments, tissue excision device 100 may be guided by and advanced through a cannula toward the ligamentum flavum 26.

In the embodiment illustrated in FIG. 4, distal cutting end 101 of tissue excision device 100 is inserted and positioned in ligamentum flavum 26 on the same side (ipsilateral) of median plane 210 as tissue excision device 100 percutaneously accesses the patient. Consequently, tissue excision device 100 does not cross median plane 210. However, in the embodiment illustrated in FIG. 5, distal cutting end 101 of tissue excision device 100 is positioned in ligamentum flavum 26 on the opposite side of median plane 210 as tissue excision device 100 percutaneously accesses the patient. Consequently, in this embodiment, tissue excision device 100 crosses median plane 210.

While it is preferred that distal cutting end 101 of tissue excision device 100 remain within ligamentum flavum 26 as shown, the presence of safety zone 40 reduces the likelihood that dural sac 32 will be damaged, even if distal tip 101 of device 100 breaks through the anterior surface of ligamentum flavum 26.

Because the present techniques are preferably performed percutaneously, certain aspects of the methods described herein may be facilitated by imaging. Imaging windows (e.g., a fluoroscopic window of access—FWA) may be employed to aid in performance of all or part of the procedures described herein. For instance, an imaging window may be employed to aid in insertion of tissue excision device 100 into ligamentum flavum 26 as shown in FIGS. 4 and 5. The methods and procedures described herein may be aided by any suitable imaging technology including, without limitation, 2D fluoroscopy, 3D fluoroscopy, CT, MRI, ultrasound or with direct visualization with fiber optic or microsurgical techniques. Stereotactic or computerized image fusion techniques are also suitable. Fluoroscopy is currently particularly well-suited to the techniques disclosed herein since fluoroscopic equipment is relatively safe and easy to use, readily available in most medical facilities, and relatively inexpensive.

In an exemplary procedure using direct biplane fluoroscopic guidance and local anesthesia, epidural space 27 is accessed for injection of contrast media adjacent to the surgical site. If the injected medium is radio-opaque, as are for example myelographic contrast media, the margins of expanded epidural space 27 will be readily visible using fluoroscopy or CT imaging. Thus, safety zone 40 created by the contrast-guided dural compression techniques can reduce the risk of damage to dural sac 32 and spinal cord 28 during MILD procedures to remove or displace portions of ligamentum flavum 26 and/or laminae 16 in order to treat spinal stenosis.

Injectable Medium

If desired, the injected fluid or medium can be provided as a re-absorbable water-soluble gel, so as to better localize safety zone 40 at the site of surgery and reduce leakage of this protective layer from the vertebral/spinal canal. The gel is preferably substantially more viscid and/or viscous than conventional contrast media. In general, a preferred viscid and/or viscous gel tends to remain localized at the desired site of treatment since it does not spread as much as standard liquid contrast media that are conventionally used in epidurography. This may result in more uniform compression of dural sac 32 and less leakage of the contrast medium out of the vertebral/spinal canal. In addition, preferred embodiments of the gel are re-absorbed more slowly than conventional contrast media, allowing for better visualization during the course of the surgical procedure(s).

A standard hydrophilic-lipophilic block copolymer (Pluronic) gel known in the art or other suitable gel may be employed as the injectable medium. The gel preferably has an inert base. In certain embodiments, the gel material is liquid at ambient temperatures and can be injected through a small bore, such as a 27 gauge needle. The gel then preferably becomes viscous when warmed to body temperature after being injected. The viscosity of the gel can be adjusted through the specifics of the preparation. The gel or other fluid is preferably sufficiently viscid and/or viscous at body temperature to compress and protect dural sac 32 in the manner described above and to remain sufficiently present in the region of interest for at least about 30 minutes. Thus, in some embodiments, the injected gel attains a viscosity that is two, three, six or even ten times that of the fluids that are typically used for epidurograms.

In certain embodiments, the injected medium undergoes a reversible change in viscosity when warmed to body temperature so that it can be injected as a low-viscosity fluid, thicken upon injection into the patient, and be returned to its low-viscosity state by cooling. In these embodiments, the injected medium is injected as desired, thickens upon warming, but can be removed by contacting it with a heat removal device, such as an aspirator that has been provided with a cooled tip. As a result of localized cooling, the gel reverts to its initial non-viscous liquid state and can be easily suctioned up the cooled needle or catheter.

In some embodiments, a contrast agent can be included in the gel itself, so that the entire gel mass is imagable. In different embodiments, the contrast agent may be injected first, followed by the desired amount of gel, or vice versa. In the embodiments in which the contrast agent and gel are injected separately, the contrast agent tends to be captured on the surface of the expanding gel mass, so that the periphery of the gel mass is imagable.

An example of a suitable injectable medium, including a contrast agent, having the desired properties is Omnipaque® 240 available from Nycomed, New York, which is a commercially available non-ionic iodinated myelographic contrast medium. Other suitable injectable media will be known to those skilled in the art. Because of the proximity to spinal cord 28 and spinal nerves 34, it is preferred not to use ionic media in the injectable medium. The preferred compositions are reabsorbed relatively rapidly after the procedure. Thus any residual gel compression on dural sac 32 after the MILD procedure dissipates relatively quickly. For example, in preferred embodiments, the gel would have sufficient viscosity to compress dural sac 32 for thirty minutes, and sufficient degradability to be substantially reabsorbed within approximately two hours.

The injected medium may further include one or more bioactive agents. For example, medications such as those used in epidural steroid injection (e.g. Depo medrol, Celestone Soluspan) may be added to the epidural gel to speed healing and reduce inflammation, scarring and adhesions. The gel preferably releases the steroid medication slowly and prolongs the anti-inflammatory effect, which can be extremely advantageous. Local anesthetic agents may also be added to the gel. This prolongs the duration of action of local anesthetic agents in the epidural space to prolong pain relief during epidural anesthesia. In this embodiment, the gel may be formulated to slow the reabsorption of the gel.

The above-described injected mediums and gels may also be used for epidural steroid injection and perineural blocks for management of acute and chronic spinal pain. Thrombin or other haemostatic agents can be added if desired, so as to reduce the risk of bleeding.

In some embodiments, the gel may also be used as a substitute for a blood patch if a CSF leak occurs. The gel may also be used as an alternative method to treat lumbar puncture complications such as post-lumbar puncture CSF leak or other causes of intracranial hypotension. Similarly, the gel may be used to patch postoperative CSF leaks or dural tears. If the dural sac were inadvertently torn or cut, then gel could immediately serve to seal the site and prevent leakage of the cerebral spinal fluid.

Ipsilateral Approach for MILD Procedure (ILAMP)

Once safety zone 40 has been created, the margins of epidural space 27 are clearly demarcated by the injected medium and may be visualized radiographically if an imageable medium or contrast agent has been used. As mentioned above, percutaneous procedures can then be performed on ligamentum flavum 26 and/or surrounding tissues, with reduced potential for injuring dural sac 32 and spinal cord 28.

A variety of suitable techniques and devices may be employed to reduce the size of the thickened/enlarged ligamentum flavum 26, thereby decompressing spinal cord 28 as well as blood vessels contained within the epidural space 27. Examples of suitable decompression techniques include without limitation, removal of tissue from ligamentum flavum 26, laminectomy, laminotomy, retraction and anchoring of ligamentum flavum 26, or combinations thereof. In some embodiments, a portion of the enlarged ligamentum flavum 26 is excised using tissue excision device 100 as best shown in FIGS. 4 and 5.

Accessing ligamentum flavum 26 with a tissue excision device 100 may present challenges. For instance, in some conventional approaches to correct stenosis caused by an enlarged ligamentum flavum 26, an incision is made in the back of the patient and then the muscles and supporting structures of the vertebral column (spine) are stripped away, exposing the posterior aspect of the vertebral column. Subsequently, the thickened ligamentum flavum 26 is exposed by removal of a portion of vertebral arch 14, often at lamina 16, which encloses the anterior portion of the spinal canal (laminectomy). The thickened ligamentum flavum 26 can then be excised by sharp dissection with a scalpel or punching instruments. However, this approach is usually performed under general anesthesia and typically requires an extended hospital stay, lengthy recovery time and significant rehabilitation. As another example, some MILD procedures access ligamentum flavum 26 percutaneously by boring a hole through the vertebral arch 14 of vertebra 10, often through a lamina 16. A cannula and/or device 100 may be passed through the bore and/or anchored to the bore to access ligamentum flavum 26 for excision. While such a MILD approach is less invasive and reduces recovery time compared to the procedure just described, such an approach requires the additional step of boring a hole in the posterior of the vertebra 10 of interest. Thus, in some cases it will be preferable to employ a MILD procedure that percutaneously accesses ligamentum flavum 26 without the need to cut or bore through the vertebra.

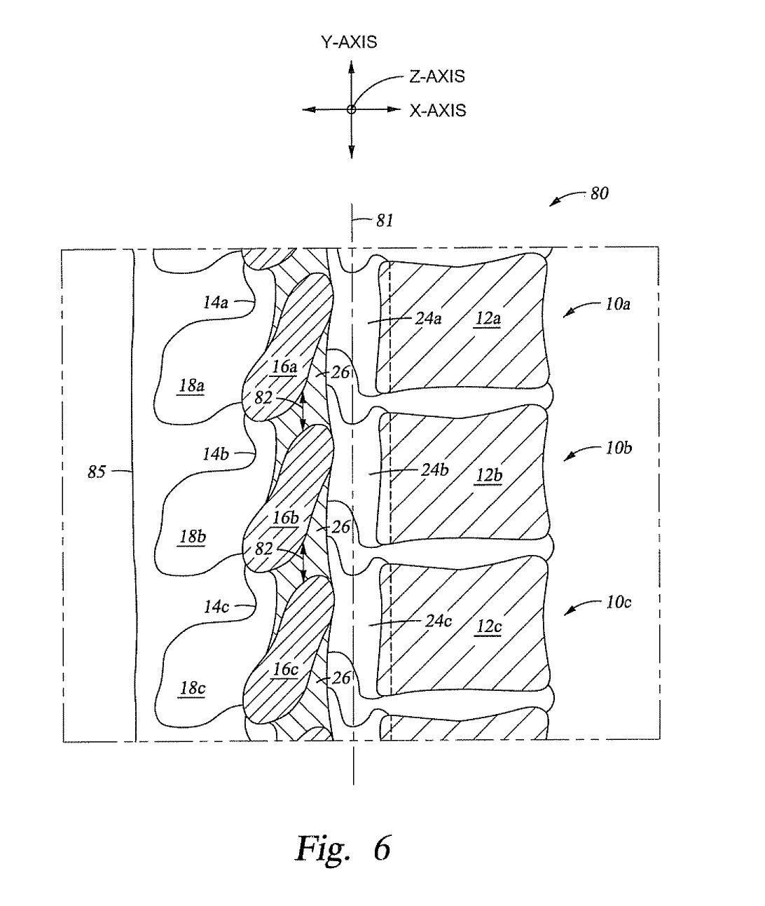

Referring now to FIG. 6, a partial cross-sectional lateral view of a segment of a vertebral column 80 is illustrated. The segment of vertebral column 80 illustrated in FIG. 6 includes three vertebrae 10a, 10b, and 10c. Each vertebrae 10a, 10b, 10c includes a vertebral body 12a, 12b, 12c, that supports a vertebral arch 14a, 14b, 14c, respectively. Vertical body 12a, 12b, 12c is anterior to vertebral arch 14a, 14b, 14c, respectively. Each vertebral arch 14a, 14b, 14c together with vertebral body 12a, 12b, 12c, respectively, encloses a vertebral foramen 15a, 15b, 15c. The succession of vertebral foramen 15a, 15b, 15c in adjacent vertebrae 10a, 10b, 10c define vertebral canal 81 (spinal canal) that runs along the length of vertebral column 80. Vertebral canal 81 contains the spinal cord (not shown in FIG. 5).

As previously described, each vertebral arch 14a, 14b, 14c includes two pedicles that project posteriorly to meet two lamina 16a, 16b, 16c, respectively. In FIG. 6, one pedicle has been removed from each vertebrae 10a, 10b, 10c and thus, only the cross-section of one lamina 16a, 16b, 16c for each vertebrae 10a, 10b, 10c, respectively, is shown. The two lamina meet posteriomedially to form the spinous process 18a, 18b, 18c, respectively.

Lamina 16a, 16b, 16c of adjacent vertebrae 10a, 10b, 10c are connected by ligamentum flavum 26 (shown in cross-section). The relatively elastic ligamentum flavum 26 extends almost vertically from superior lamina to inferior lamina of adjacent vertebrae. In particular, ligamentum flavum 26 originates on the inferior surface of the laminae of the superior vertebra and connects to the superior surface of the laminae of the inferior vertebra. For instance, ligamentum flavum 26 originates on the inferior surface of lamina 16a of superior vertebra 10a and connects to the superior surface of lamina 16b of the inferior vertebra 10b. Thus, ligamentum flavum 26 spans an interlaminar space 82. Interlaminar space 82 is generally the space between laminae of adjacent vertebrae in spinal column 80.

Still referring to FIG. 6, each lamina 16a, 16b, 16c comprises a relatively broad flat plate of bone that extends posteromedially and slightly inferiorly from pedicles 24a, 24b, 24c, respectively. Along the length of vertebral column 80, the lamina 16a, 16b, 16c overlap like roofing shingles, with each lamina substantially parallel to and at least partially overlapping the adjacent inferior lamina. Further, the adjacent substantially parallel laminae are separated by the intervening ligamentum flavum 26 and interlaminar space 82. For instance, lamina 16a is substantially parallel to and partially overlaps adjacent inferior lamina 16b and is separated from lamina 16b by ligamentum flavum 26 and interlaminar space 82.

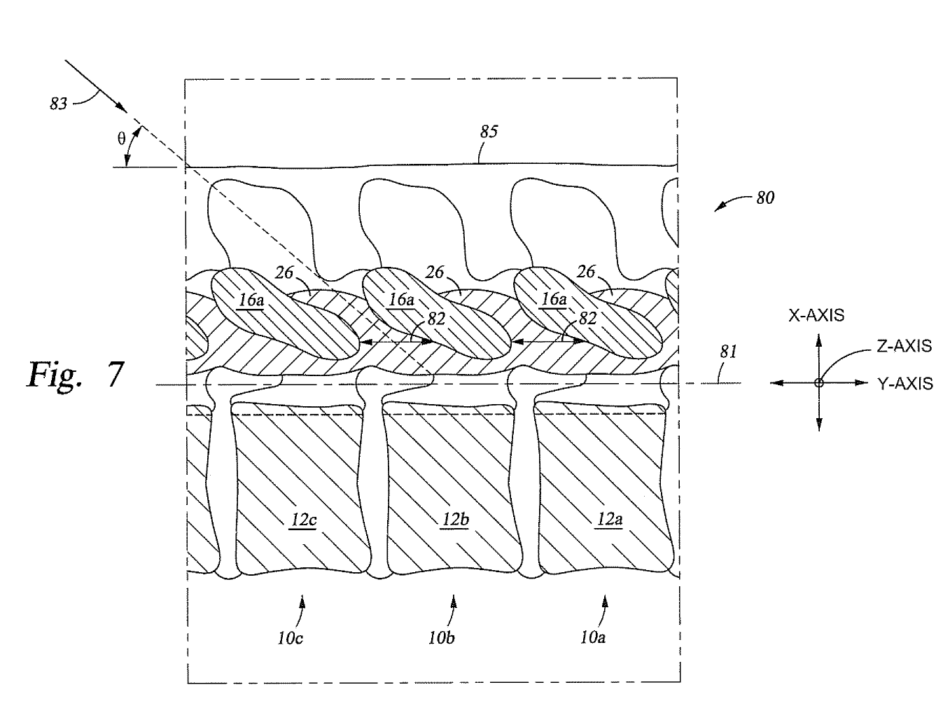

FIG. 7 illustrates vertebral column 80 as it may be oriented with the anterior side positioned down and posterior back surface 85 positioned upward, as may be encountered during a spinal procedure or surgery. In addition, in the embodiment illustrated in FIG. 7, ligamentum flavum 26 is thickened or enlarged, resulting in spinal stenosis. In particular, the anterior portions of enlarged ligamentum flavum 26 extend partially into spinal canal 81, potentially exerting compressive forces on the spinal cord (not shown) that resides within spinal canal 81.

As previously discussed, to relieve compressive forces on the spinal cord and hence relieve the associated symptoms of spinal stenosis, portions of ligamentum flavum 26 may be excised. However, to percutaneously excise portions of ligamentum flavum 26 via minimally invasive techniques, the innate structure of vertebral column 80 and each vertebra may present significant imaging challenges. For instance, lateral imaging windows/views of ligamentum flavum 26 substantially in the direction of the z-axis may be obscured by the various processes of the vertebrae (e.g., transverse processes, superior articular processes, inferior articular processes), the laminae of the vertebra, etc. Further, some anterior-posterior (A-P) imaging windows/views of ligamentum flavum 26 substantially in the direction of the x-axis may also be obscured by the laminae. In particular, in the A-P radiographic imaging planes substantially in the direction of the x-axis, the posterior edges of parallel laminae overlap and obscure ligamentum flavum 26 and interlaminar space 82, particularly the anterior portions of ligamentum flavum 26 and interlaminar space 82 closest to spinal canal 81. However, with an imaging window/view in a plane substantially parallel to the X-Y plane, at an angle θ generally in the direction of arrow 83, and slightly lateral to the spinous process, interlaminar space 82 and ligamentum flavum 26 may be viewed with less obstruction from neighboring laminae. In other words, imaging windows/views generally aligned with arrow 83 (FIG. 7) allow a more direct view of interlaminar space 82 and ligamentum flavum 26 from the posterior back surface with minimal obstruction by the vertebrae, and more specifically the laminae.

Typically, the long axes of the substantially parallel laminae (e.g., laminae 16a, 16,b, 16c) and interlaminar spaces (e.g, interlaminar spaces 82) are generally oriented between 60° and 75° relative to posterior back surface 85. Thus, preferably the imaging means (e.g., x-ray beam, fluoroscopy tube, etc.) is positioned generally in the direction represented by arrow 83, where θ between posterior back surface 85 and the imaging beam is substantially between 60° and 75°. In other words, the imaging means is positioned substantially parallel to the surface of the laminae. The resulting imaging window/view, termed “caudal-cranial posterior view” hereinafter, permits a clearer, more direct, less obstructed view of interlaminar space 82 and ligamentum flavum 26 from the general posterior back surface 85. The caudal-cranial posterior view permits a relatively clear view of interlaminar space 82 and ligamentum flavum 26 in directions generally along the y-axis and z-axis. However, the caudal-cranial posterior view by itself may not provide a clear imaging window/view of interlaminar space 82 and ligamentum flavum 26 in directions generally along the x-axis. In other words, the caudal-cranial posterior view by itself may not provide a clear imaging window or view that can be used to accurately determine the posterior-anterior depth, measured generally along the x-axis, of a device across the ligamentum flavum 26.

Thus, in preferred embodiments, an additional imaging window/view, termed “caudal-cranial posterior-lateral view” hereinafter, is employed to provide a clearer, unobstructed view of interlaminar space 82 and ligamentum flavum 26 in directions generally along the y-axis and z-axis. The caudal-cranial posterior-lateral view is generated by orienting an imaging means generally at an angle θ relative to posterior back surface 85 of the patient and also angling such imaging means laterally in an oblique orientation, revealing a partial lateral view of interlaminar space 82 occupied by ligamentum flavum 26 on the anterior side of the lamina and posterior to the underlying dural sac (not shown) and spinal cord (not shown).

By employing at least one of the caudal-cranial posterior view and the caudal-cranial posterior-lateral views, relatively clear imaging windows/views of the interlaminar space 82 and ligamentum flavum 26 in directions along the x-, y-, and z-axes may be achieved.

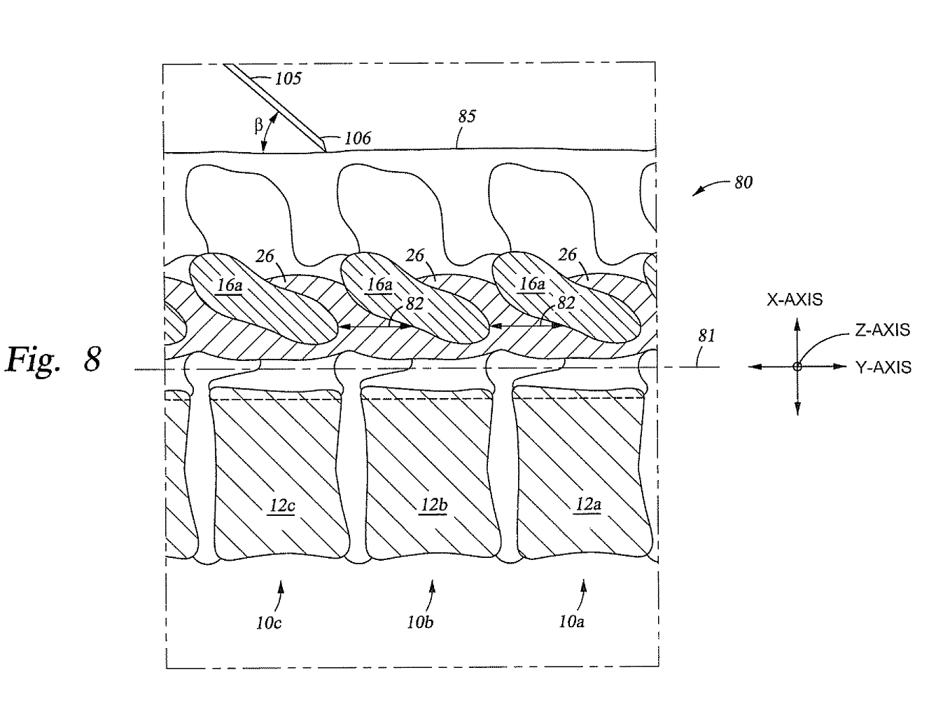

Referring now to FIG. 8, vertebral column 80 and a tissue access instrument 105 including a distal end 106 are illustrated. Tissue access instrument 105 may comprise a tissue excision device (e.g., tissue excision device 100), a cannula, a catheter, or other portal. Once unobstructed imaging windows/views of interlaminar space 82 and ligamentum flavum 26 are established in the manner previously described, tissue access instrument 105 is employed to percutaneously access interlaminar space 82 and ligamentum flavum 26. More specifically, using images of the interlaminar space 82 and ligamentum flavum 26 obtained from the desired direction(s), (e.g., caudal-cranial posterior view and the caudal-cranial posterior-lateral view), tissue access device 105 may be employed to penetrate the skin and soft tissue in the posterior back surface 85 of the patient. In preferred embodiments, the skin entry point for tissue excision device 100 is between 5 and 10 cm inferior (caudal to) the posterior surface of the interlaminar space 82 of interest. For instance, if the portion of ligamentum flavum 26 between lamina 16a and lamina 16b is the area of interest, then tissue excision device 100 may be inserted into the patient's back about 5 to 10 cm inferior to posterior surface 84 of interlaminar space 82.

Referring still to FIG. 8, tissue access device 105 is preferably initially inserted into the posterior tissue and musculature of the patient generally parallel to the longitudinal axis of spinal column 80. In other words, the angle β between the posterior back surface 85 and tissue access device 105 is preferably between 0° and 10° when tissue access device 105 is initially inserted. Further, tissue access device 105 is preferably inserted into the posterior tissue and musculature of the patient on the same side (ipsilateral) of the median plane as the area of interest (e.g., the targeted portion of ligamentum flavum 26), as best seen in FIG. 4. Once tissue access device 105 is inserted into the posterior tissue and musculature of the patient, tissue access device 105 then may be oriented 5° to 90° relative to the posterior back surface 85 in order to create a trajectory across ligamentum flavum 26 in the area of interest. It is to be understood that once tissue access device 105 is inserted into the patient's posterior back surface 85, the ends of tissue access device 105 (e.g., distal end 106) are free to pivot about the insertion location in posterior back surface 85 in the general direction of the y-axis and the z-axis, and may be advanced posteriorly or anteriorly generally in the direction of the x-axis.

Once inserted into the posterior tissue and musculature of the patient, tissue access device 105 can be positioned to provide a trajectory across interlaminar space 82 in the area of interest, generally towards the anterior surface of the lamina superior to the area of interest. For example, if interlaminar space 82 between lamina 16a and lamina 16b is the area of interest, tissue access device 105 is positioned to provide a trajectory that will allow a cutting instrument to be inserted across interlaminar space 82 between lamina 16a and lamina 16b towards the anterior surface of lamina 16a (superior lamina).

By switching between the caudal-cranial posterior view and the caudal-cranial posterior-lateral view, or by viewing both the caudal-cranial posterior view and the caudal-cranial posterior-lateral view at the same time, tissue access device 105 can be advanced to ligamentum flavum 26 in the area of interest with more certainty than has heretofore been present. Once distal end 106 of tissue access device 105 has reached ligamentum flavum 26, portions of ligamentum flavum 26 may be excised with a tissue excision device (e.g., tissue excision device 100) so as to relieve pressure on the spinal nerves. If tissue access device 105 comprises a tissue excision tool, it may be inserted into ligamentum flavum 26 to excise portions of ligamentum flavum 26. However, if tissue access device 105 comprises a cannula or portal, tissue access device 105 will be positioned adjacent or slightly within the ligamentum flavum 26 in the region of interest and a tissue excision device may be advanced through, and guided by, tissue access device 105 toward ligamentum flavum 26. In some embodiments, excision can be performed generally from posterior to anterior across interlaminar space 82 and then laterally along the anterior portion of ligamentum flavum 26 if desired. The actual depth of distal end 106 of tissue access device 105 (or any tissue excision device passing through tissue access device 105) in the general direction of the x-axis may be adjusted with guidance from the caudal-cranial posterior-lateral view and appropriate retraction/advancement of tissue access device 105 and appropriate adjustment of tissue access device 105 between 5° and 90° relative to the posterior back surface 85.

Referring now to FIG. 4, the tip of an exemplary tissue excision device 100 is shown schematically within ligamentum flavum 26. Tissue excision device 100 may be the same device as tissue access device 105, or may be a tool passed through tissue access device 105 if tissue access device 105 is a cannula or portal. In particular, tissue excision device 100 has accessed ligamentum flavum 26 according to the ILAMP method previously described. Thus, device 100 is positioned to excise portions of ligamentum flavum 26 on the same lateral side of median plane 210 as device 100 is percutaneously inserted. In other words, in the view shown in FIG. 4, device 100 is inserted into the body on the right side of median plane 210 and enters ligamentum flavum 26 on the right side of median plane 210 to excise portions of ligamentum flavum 26 on the right side of median plane 210. In FIG. 4, device 100 does not cross median plane 210.

FIG. 5 illustrates an embodiment of an alternative MILD method in which exemplary tissue excision device 100 is positioned to excise portions of ligamentum flavum 26 on the opposite lateral side of median plane 210 as device 100 is percutaneously inserted. More specifically, tissue excision device 100 is inserted into the body on the rights side of median plane 210, enters ligamentum flavum 26 on the right side of median plane 210, but is positioned to excise portions of ligamentum flavum 26 on the left side of median plane 210. In FIG. 5, device 100 crosses median plane 210.

In the manner described, portions of the ligamentum flavum can be excised by a percutaneous MILD procedure. In particular, with the approach described and as best illustrated in FIGS. 4 and 6, ligamentum flavum 26 can be accessed, and portions thereof removed via the interlaminar space on the same lateral side (ipsilateral) of median plane 210 as the entry point for instrument 101 (e.g., a cannula, a tissue excision tool, etc.). This approach may sometimes hereinafter be referred to as an Iplsilateral Approach MILD Procedure (ILAMP).

Tissue Excision Devices and Methods

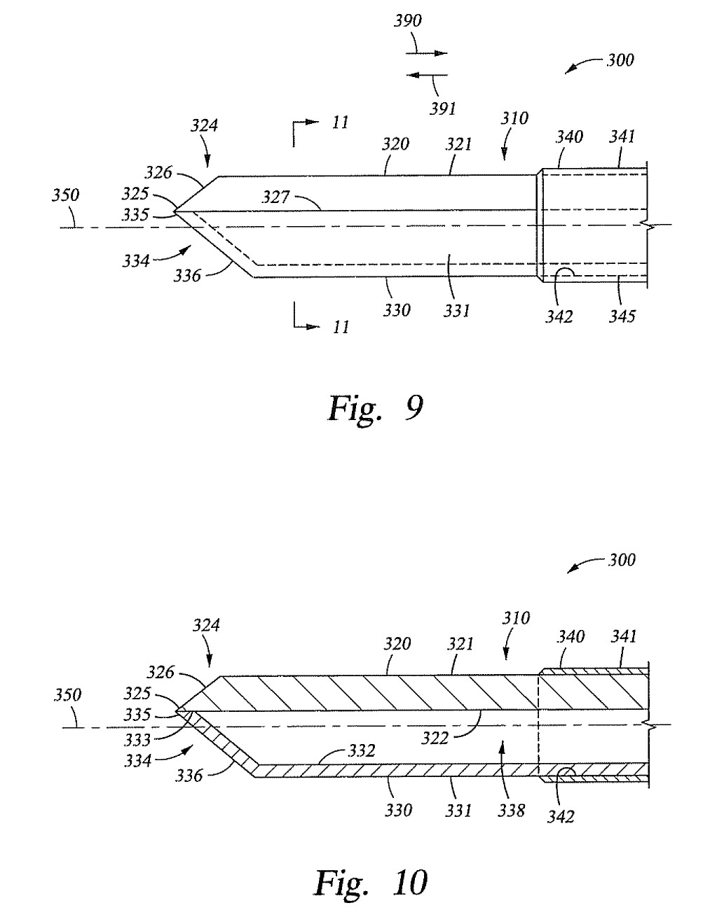

Referring now to FIGS. 9-11, the distal portion of an embodiment of a tissue excision device 300 is illustrated. As used herein, the term “distal” refers to positions or portions of a device that are relatively closer to the region of interest (e.g., the thickened portion of the ligamentum flavum to be decompressed), and relatively further from the user of the device (e.g., a surgeon). Tissue excision device 300 comprises an elongate body 310 having a longitudinal axis 250 and including an elongate first or upper member 320 that slidingly engages an elongate second or lower member 330.

Upper member 320 comprises a distal end 324, an inner surface 322 that generally faces lower member 330, and an outer surface 321 generally opposite inner surface 322 and facing away from lower member 330. Inner surface 321 and outer surface 322 intersect to form an edge 327. Inner surface 322 of upper member 320 slidingly engages lower member 330. In addition, distal end 324 includes a cutting tip 325 that is sharpened via a beveled surface 326 extending between inner surface 322 and outer surface 321. Sharpened cutting tip 325 enhances the ability of distal end 324 of upper member 320 to cut or slice through tissue. Thus, distal end 324 may be described herein as a “distal cutting end.” In this embodiment, inner surface 321 is substantially planar and substantially parallel to axis 350. However, in different embodiments, inner surface 321 may be arcuate and/or not parallel with axis 350.

Likewise, lower member 330 comprises a distal end 334, an inner surface 332 that faces upper member 320, and an outer surface 331 generally opposite inner surface 332 and facing away from upper member 320. In addition, lower member 330 includes an inner hollow region or cavity 338 defined by inner surface 332. Cavity 338 is open to, and generally faces, upper member 320. As will be explained in more detail below, segments of tissue excised by tissue excision device 300 are at least temporarily held within cavity 338, and thus, cavity 338 may be referred to herein as a “tissue capture chamber.” Although the embodiments illustrated herein show lower member 330 including cavity 338, while upper member 320 does not include a cavity or tissue capture chamber, it should be appreciated that in other embodiments, upper member 320 and/or lower member 330 may include a cavity or tissue capture chamber.

Referring still to FIGS. 9-11, a dynamic sliding surface 333 extends between outer surface 331 and inner surface 332 of lower member 330. Dynamic sliding surface 333 slidingly engages the outer peripheral portions of inner surface 322 of upper member 320. In this embodiment, distal end 334 of lower member 330 includes a cutting tip 335 that is sharpened via a beveled surface 336 extending between inner outer surface 331 and dynamic sliding surface 333. Sharpened cutting tip 335 enhances the ability of distal cutting end 334 of lower member 330 to cut or slice through tissue. Thus, distal end 334 may be described herein as a “distal cutting end.” In some embodiments, the distal end of lower member 330 may not be sharpened.

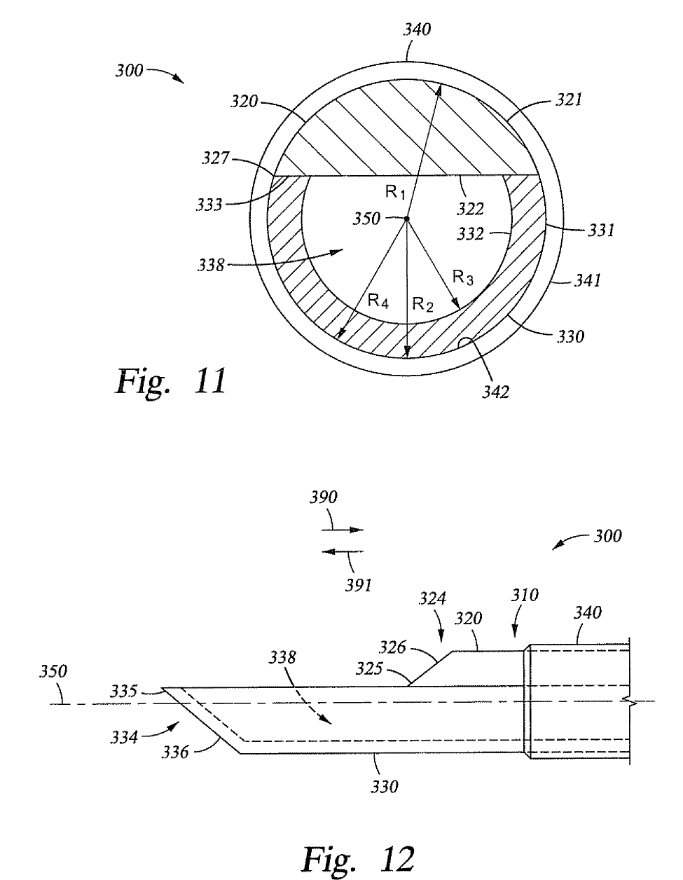

In this embodiment, body 310 has a generally circular cross-section as best seen in FIG. 11. Specifically, outer surface 321 of upper member 320 is positioned at a radius R₁, outer surface 331 of lower member 330 is positioned at a radius R₂ that is substantially the same as radius R₁. Thus, outer surface 321 of upper member 320 and outer surface 331 of lower member 330 meet to form a substantially cylindrical body 310. Further, inner surface 332 of lower member 330 is positioned substantially at a radius R₃ that is less than R₁ and R₂. Thus, in this embodiment, cavity 338 has a semi-circular cross-section. Although body 310 and cavity 338 illustrated herein have circular or semi-circular cross-sections, it should be appreciated that in general, body 310 and/or cavity 338 may have any suitable cross-sectional shape including, without limitation, rectangular, triangular, oval, or polygonal.

As previously described, dynamic sliding surface 333 of lower member 330 slidingly engages the outer periphery of inner surface 321 of upper member 320. Thus, upper member 320 and lower member 330 may move relative to each other. In particular, upper member 320 and lower member 330 may move in directions substantially parallel to each other, generally in the directions of arrows 390, 390. Further, in this embodiment, upper member 320 and lower member 330 may each move in directions substantially parallel to axis 350. For example, upper member 320 and lower member 330 may move axially with respect to axis 350. However, upper member 320 and lower member 330 are restricted from moving in directions other than parallel to each other and axis 350. For instance, in this embodiment, upper member 320 and lower member 330 are restricted from moving radially towards or away from axis 350.

In the embodiment illustrated in FIGS. 9-11, the sliding engagement and movement of upper member 320 and lower member 330 are restricted to directions substantially parallel with each other and axis 350 by a sleeve 340. Specifically, sleeve 340 includes an outer surface 341 and an inner surface 342 defining a through bore 345. Body 310 is disposed within bore 345 of sleeve 340 with the distal portions of upper member 320 and lower member 330 extending therefrom. Outer surfaces 321, 331 of members 320, 330 slidingly engage inner surface 342 of sleeve 340. In this embodiment, inner surface 342 of sleeve 340 is positioned at a radius R₄ that is the same or slightly greater than radii R₁, R₂ of outer surfaces 321, 331, respectively. Thus, since body 310 has a circular cross-section in this embodiment, inner surface 342 of sleeve 340 has a mating circular cross-section. However, it should be appreciated that inner surface 342 of sleeve 340 may have any suitable cross-section that mates with the outer surfaces 321, 331 thereby restricting movement of upper member 320 relative to lower member 330 in direction other than parallel to each other and axis 350.

As described above, upper member 320 and lower member 330 are permitted to move substantially parallel to each other and axis 350 (i.e., generally in the direction of arrows 390, 391), but are restricted from moving in other directions by sleeve 340. However, it should be appreciated that other suitable means may be employed to limit the relative movement of upper member 320 and lower member 330. For instance, in addition to, or as an alternative to sleeve 340, a mating track or rail system may be employed between upper member 320 and lower member 330 to limit their relative motion to directions parallel to each other and/or axis 350. Such a track or rail system may be positioned between and along dynamic sliding surface 333 of lower member 330 and the outer periphery of upper member 320.

Referring still to FIGS. 9-11, in some embodiments, the proximal end of tissue excision device 300 (not shown) is coupled to a handle to facilitate control and movement of tissue excision device 300. The handle may be constructed from any suitable material including without limitation machined metal or molded from plastic. In addition, in other embodiments, the proximal end of body 310 and/or sleeve 340 may be coupled to an actuation means that enables controlled movement of upper member 320, lower member 330, sleeve 340, or combinations thereof relative to each other. A suitable actuation means is disclosed in U.S. application Ser. No. 11/461,045 entitled “Percutaneous Tissue Excision Devices and Methods,” which is hereby incorporated herein by reference in its entirety.

Referring now to FIGS. 9 and 12, tissue excision device 300 has a first or opened position (FIG. 12) and a second or closed position (FIG. 9) depending on the position of upper member 320 relative to lower member 330. When tissue excision device 300 is in the closed position, upper member 320 is disposed across the opening to cavity 338 and distal ends 324, 334 are substantially adjacent each other. Thus, when tissue excision device 300 is in the closed position, upper member 320 closes off and covers cavity 338. In addition, when tissue excision device 300 is in the closed position, upper member 320 and lower member 330 do not extend distally beyond one another. However, when tissue excision device 300 is in an opened position, upper member 320 is not disposed across cavity 338 and distal ends 324, 334 are spaced apart. Specifically, lower member 330 extends from upper member 320 and the opening of cavity 338 is not covered or closed off by upper member 320. Thus, when tissue excision device 300 is in an opened position, cavity 338 is open to receive excised tissue (e.g., cavity 338 is open to the environment outside tissue excision device 300).

Referring still to FIGS. 9 and 12, tissue excision device 300 is transitioned between the closed position (FIG. 9) and the opened position (FIG. 12) by moving upper member 320 and lower member 330 relative to each other. Specifically, in the embodiments illustrated herein, upper member 320 is slid in the direction of arrow 390 and retracted into sleeve 340, thereby opening cavity 338 to the environment external to tissue excision device 300. Alternatively, in different embodiments, lower member 330 is slid in the direction of arrow 391 and extended further from sleeve 340, thereby opening cavity 338 to the environment external to tissue excision device 300.

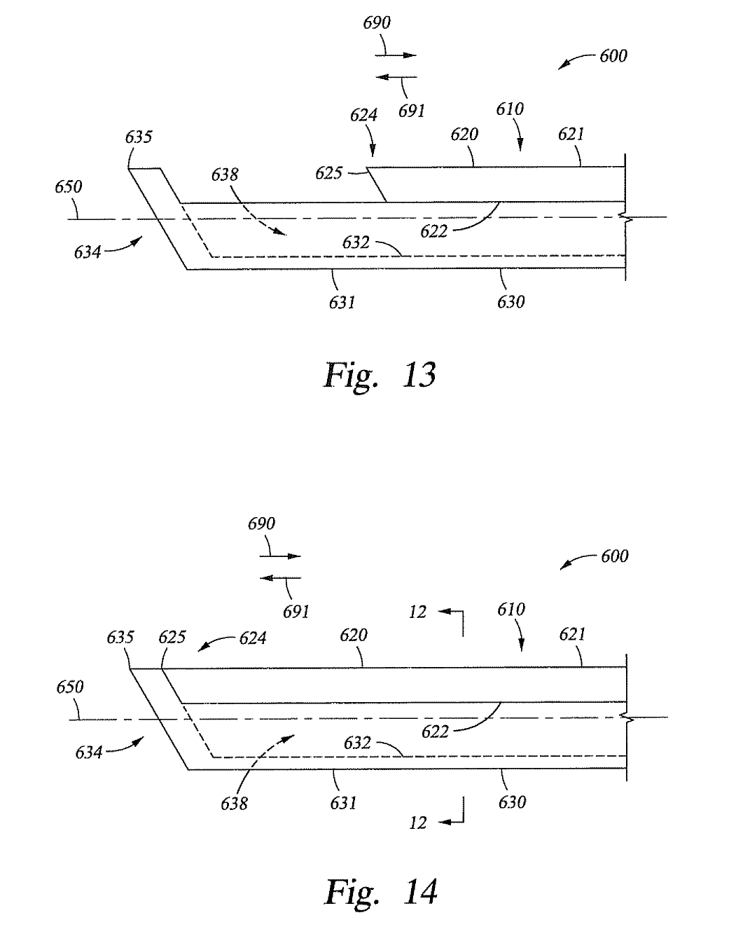

Referring now to FIGS. 13-15, the distal portion of another embodiment of a tissue excision device 600 is illustrated. Tissue excision device 600 comprises an elongate body 610 having a longitudinal axis 650 and including an elongate first or upper member 620 that slidingly engages an elongate second or lower member 630.

Upper member 620 comprises a distal end 624, an inner surface 622 that generally faces lower member 630, and an outer surface 621 generally opposite inner surface 622 and facing away from lower member 630. Distal end 624 includes a sharpened cutting tip 625. Sharpened cutting tip 625 enhances the ability of distal end 624 to cut or slice through tissue. Thus, distal end 624 may be described herein as a “distal cutting end.”

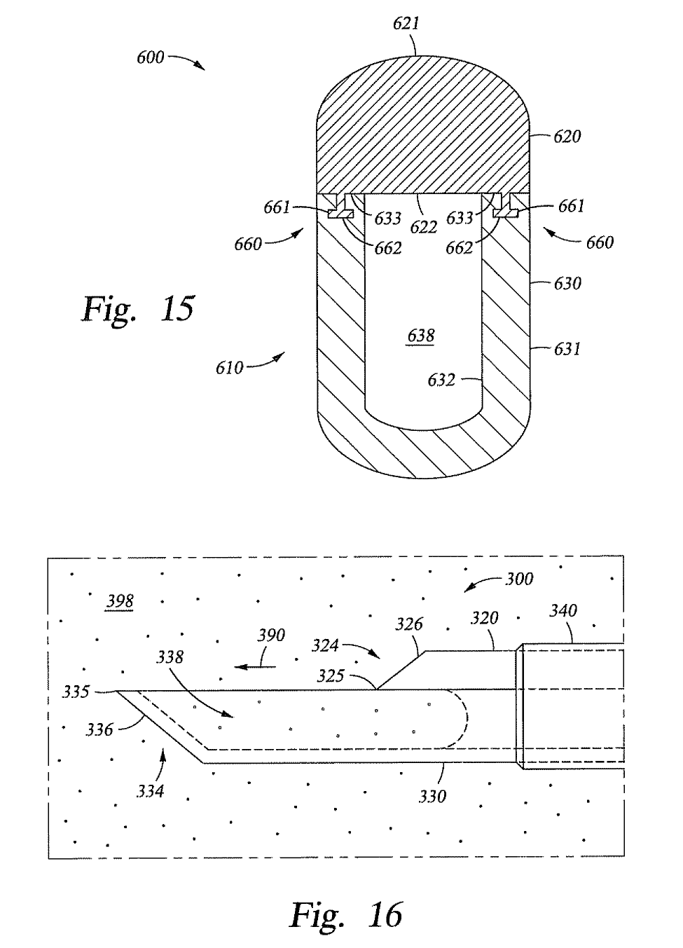

Likewise, lower member 630 comprises a distal end 634, an inner surface 632 that faces upper member 620, an outer surface 631 generally opposite inner surface 632 and facing away from upper member 620, and a dynamic sliding surface 633 extending between inner surface 632 and outer surface 631. In addition, lower member 630 includes an inner hollow region or cavity 638 defined by inner surface 632. Cavity 638 is open to, and generally faces, upper member 620. Since tissue excised by tissue excision device 600 is held within cavity 638, cavity 638 may be referred to herein as a “tissue capture chamber.” Distal end 634 of lower member 630 includes a cutting tip 635. In some embodiments, distal end 634 may be sharpened. In this embodiment, body 610 does not have a circular cross-section, but rather more of rectangular cross-section with rounded corner as best seen in FIG. 15.

Referring specifically to FIGS. 13 and 14, tissue excision device 600 operates substantially the same as tissue excision device 300 previously described. Namely, tissue excision device 600 has a first or opened position (FIG. 13) and a second or closed position (FIG. 14) depending on the position of upper member 620 relative to lower member 630. When tissue excision device 600 is in the closed position, upper member 620 is disposed across the opening to cavity 638 and distal ends 624, 634 are substantially adjacent each other. Thus, when tissue excision device 600 is in the closed position, upper member 620 closes off and covers cavity 638. However, when tissue excision device 600 is in an opened position, upper member 620 is not disposed across cavity 638 and distal ends 624, 634 are spaced apart. Specifically, lower member 630 extends from upper member 620 and the opening of cavity 638 is not covered or closed off by upper member 620. Tissue excision device 600 is transitioned between the closed position (FIG. 14) and the opened position (FIG. 13) by moving upper member 620 and lower member 630 relative to each other in directions substantially parallel to each other and axis 650 (i.e., in the direction of arrows 690, 691).

Referring now to FIG. 15, rather than employing a sleeve (e.g., sleeve 340) to restrict the relative motion of upper member 620 and lower member 630, tissue excision device 600 includes a rail or track system 660 that permits the movement of upper member 620 relative to lower member 630 in directions substantially parallel to members 620, 630 and axis 650. However, rail system 660 restricts the motion of upper member 620 relative to lower member 630 in all other directions. In the embodiment illustrated in FIG. 15, upper member 620 comprises a projection 661 that mates with a recess 662 provided in dynamic sliding surface. Specifically, projection 661 has a T-shaped cross-section that slidingly engages the mating T-shaped recess 662.

Tissue Excision Methods

FIGS. 16-18 schematically illustrate the excision of a segment of tissue 398 by tissue excision device 300 previously described. In some embodiments, a portal or cannula (not shown) may be employed to provide tissue excision device 300 percutaneous access to tissue 398. For instance, tissue excision device 300 may be inserted into and advanced through such a portal or cannula to reach tissue 398. If a portal or cannula is used to guide tissue excision device 300, tissue excision device 300 may be passed through such cannula in the opened position or closed position. Several exemplary tools, devices and methods employing a portal to provide percutaneous access to a tissue of interest are disclosed in U.S. application Ser. No. 11/461,020, which is hereby incorporated herein by reference in its entirety.

Regardless of the manner in which tissue excision device 300 reaches the tissue 398 (e.g., by portal or otherwise), prior to insertion into the tissue to be excised (e.g., tissue 398), tissue excision device 300 is configured in the opened position as shown in FIG. 12. Tissue excision device 300 is advanced into tissue 398 in the opened position and with distal cutting ends 324, 334 first. Tissue excision device 300 is advanced generally in the direction of arrow 390, substantially parallel to axis 350 as best shown in FIG. 16. Tissue 398 may be any type of tissue to be excised and removed from a patient including without limitation, soft tissue, fat, muscle, or combinations thereof. When used to treat spinal stenosis caused by a thickened ligamentum flavum, the distal portion of tissue excision device 300 is preferably inserted into the stenotic ligamentum flavum 26, preferably posterior to a safety zone 40, in order to safely cut and remove portions of the thickened ligamentum flavum 26 (see FIGS. 2 and 3), thereby reducing the stenosis.

Still referring to FIGS. 16-18, as tissue excision device 300 is inserted and advanced into tissue 398, cutting tips 325, 335 cut through tissue 398. In addition, as tissue excision device 300 is advanced into tissue 398, portions of tissue 398 cut by cutting tips 325, 335 slide into and fill at least a portion of cavity or tissue capture chamber 338 of lower member 330. It is to be understood that the farther tissue excision device 300 is advanced into tissue 398, the greater the amount of tissue 398 that is cut, and the greater the amount of excised tissue 398 that will occupy tissue capture chamber 338. Upper member 320 and lower member 330 are preferably sufficiently rigid such that they do not flex or bend significantly as they are advanced through tissue 398.

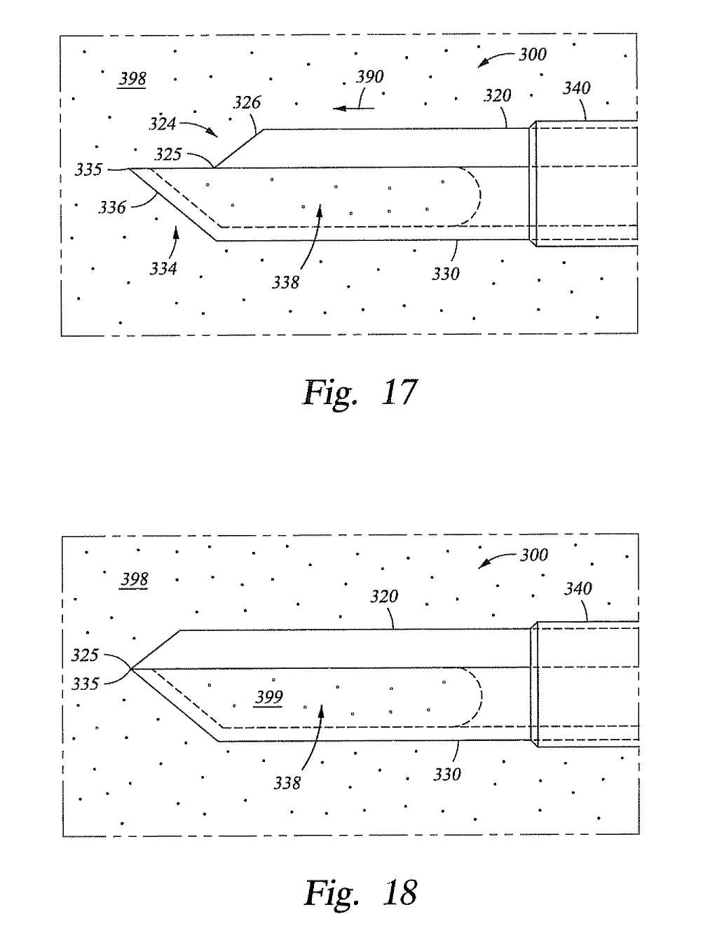

Once the desired amount of tissue 398 has been excised and captured in tissue capture chamber 338, tissue excision device 300 may be transitioned to the closed position as best seen in FIGS. 17 and 18. Specifically, upper member 320 is slid in the direction of arrow 390 relative to lower member 330, thereby transitioning tissue excision device 300. As upper member 320 is slid relative to lower member 330 in the direction of arrow 390, the segment of tissue 398 within tissue capture chamber 338 is severed from the surrounding tissue 398 as best seen in FIG. 17. In particular, sharpened cutting tip 325 of upper member 320 slices tissue extending outside tissue capture chamber 338. Upper member 320 is advanced substantially parallel to axis 350 in the direction of arrow 390 until cutting tips 325, 335 meet as best seen in FIG. 18. Once tissue excision device 300 has achieved the closed position shown in FIG. 18, the excised tissue segment 399 (i.e., the excised segment of tissue 398) within tissue capture chamber 338 is completely separated from the remaining tissue 398 external to tissue excision device 300.

At this point, tissue excision device 300, along with excised tissue segment 399 contained within tissue capture chamber 338, is retracted from tissue 398. Once tissue excision device 300 has been completely removed from the patient, tissue excision device 300 is transitioned to an opened position so that excised tissue segment may be removed from tissue capture chamber 338. Once tissue capture chamber 338 has been emptied, manually or otherwise, tissue excision device 300 may be reinserted into tissue 398 to continue to the cutting and removal of portions of tissue 398.

Excised tissue segment(s) 399 held within tissue capture chamber 338 may be removed by simply opening tissue excision device 300 and pulling the excised tissue segments 399 from tissue capture chamber 338. In alternative embodiments described in more detail below, excised tissue segment(s) 399 may be emptied and removed from tissue capture chamber 338 by a tissue ejector included within tissue excision device 300. Still further, in other embodiments described in more detail below, excised tissue segment(s) 399 may be retrieved and removed from tissue capture chamber 338 without the need to withdraw, remove, or reposition tissue excision device 300 within tissue 398.

Tissue Removal and Retrieval

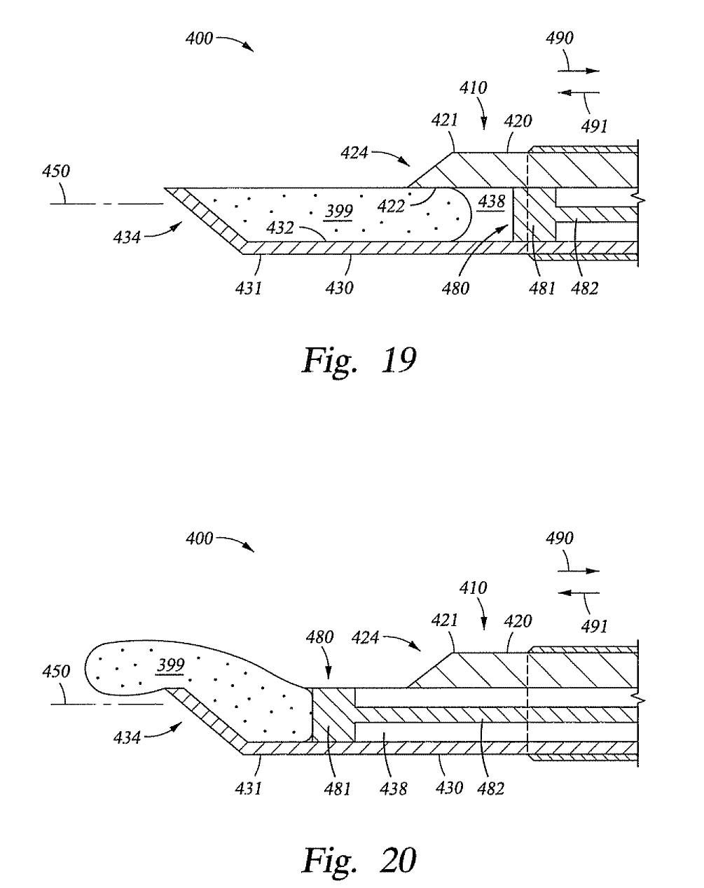

Referring briefly to FIGS. 19 and 20, the distal portion of another embodiment of a tissue excision device 400 is illustrated. Similar to tissue excision device 300 described above, tissue excision device 400 comprises an elongate body 410 having a longitudinal axis 250 and including an elongate upper member 420 that slidingly engages an elongate lower member 430. Upper member 420 comprises a distal cutting end 424, an outer surface 421 and an inner surface 422. Likewise, lower member 430 comprises a distal cutting end 434, an outer surface 431 and an inner surface 432. In addition, lower member 430 includes an inner hollow region or cavity 438 defined by inner surface 432. Excised tissue segment 399 is temporarily held within cavity 438, and thus, cavity 438 may be referred to herein as a “tissue capture chamber.”

Tissue excision device 400 functions substantially the same as tissue excision device 300 previously described. For instance, tissue excision device 400 has an opened position (FIG. 19) in which distal cutting end 434 of lower member 430 extends from upper member 420 and tissue capture chamber 438 is open to the environment external tissue excision device 400; and further, tissue excision device 400 has a closed position (not shown) in which distal cutting ends 424, 434 are adjacent each other and tissue capture chamber 438 is closed off by upper member 420. However, tissue excision device 400 further comprises a tissue ejector 480 slidingly disposed within cavity or tissue capture chamber 438.

Referring still to FIGS. 19 and 20, tissue ejector 480 includes a plunger 481 coupled to an ejection shaft 482. Plunger 481 and ejection shaft 482 slide axially within cavity 438 substantially parallel to axis 450 and generally in the direction of arrows 490, 491. Plunger 481 is shaped and configured to fit within tissue capture chamber 438 between upper member 420 and lower member 430. Further, plunger 481 preferably slidingly contacts inner surface 422 of upper member 420 and inner surface 432 of lower member 430.

Once tissue excision device 400 has excised tissue segment 399 in the manner previously described and tissue excision device 400 has been completely removed from the patient, excised tissue segment 399 is removed or emptied from tissue capture chamber 438 by ejector 480 Specifically, with tissue excision device 400 in the opened position (FIG. 19), excised tissue segment 399 within tissue capture chamber 438 is removed from tissue excision device 400 by advancing ejector shaft 482 and plunger 481 in the direction of arrow 491 toward excised tissue segment 399. As plunger 481 engages excised tissue segment 399, plunger 481 will urge excised tissue segment 399 generally in the direction of arrow 491 and out of tissue capture chamber 438 (FIG. 20), thereby ejecting excised tissue segment 399 from tissue excision device 400.

In some embodiments, ejector 480 may be controlled by a multi-function tool. Embodiments of suitable multi-function tools for ejecting a tissue segment from a tissue retrieval device (e.g., tissue retrieval device 400) are disclosed in U.S. application Ser. No. 11/461,045, which is hereby incorporated herein by reference in its entirety.

In one specific application, embodiments of the tissue excision devices described herein (e.g., tissue excision device 300, 400) are employed to excise relatively small portions of the stenosed or enlarged ligamentum flavum. By excising several small portions of the ligamentum flavum, the enlarged ligamentum flavum may be decompressed, thereby relieving pressure imposed on the spinal cord and the associated pain and other symptoms. Since the surgical procedures described herein are performed adjacent sensitive tissue (e.g., nerves of the spinal cord), they are preferably performed delicately and with minimal movement of the tools and devices near the sensitive tissues. Thus, it may be desirable to minimize repositioning of the tissue excision device, especially the distal tip or cutting end of the tissue excision device during the excision procedures. For example, it may be advantageous to maintain the tissue excision device substantially within or adjacent the area of interest (e.g., enlarged ligamentum flavum) while making repeated excisions of portions of the tissue in the region of interest. In other words, it may be preferred that the tissue excision device not be completely withdrawn from the area of interest and reinserted into the area of interest between each separate excision. However, in cases when the distal tip or cutting end of the tissue excision device is maintained within the area of interest (i.e., not removed from the patient between each excision), the excised tissue segments may build up within the tissue capture chamber (e.g., cavity 438) of the tissue excision device. Excessive build-up of excised tissue within the tissue excision device may inhibit or detrimentally impact continued cutting. Thus, between each excision by the tissue excision device, or at any desired time or interval, excised tissue segments within the tissue capture chamber of the tissue excision device are preferably retrieved and removed without necessitating the removal of the tissue excision device itself.

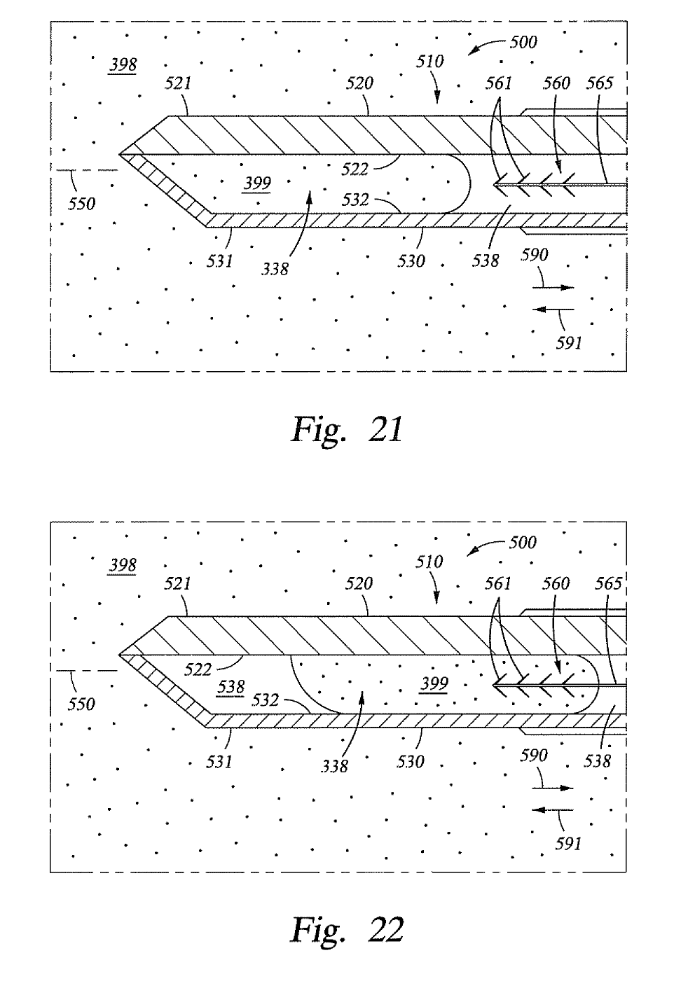

Referring now to FIGS. 21 and 22, the distal portion of another embodiment of a tissue excision device 500 is illustrated. Similar to tissue excision device 300 described above, tissue excision device 500 comprises an elongate body 510 having a longitudinal axis 550 and including an elongate upper member 520 that slidingly engages an elongate lower member 530. Upper member 520 comprises an outer surface 521 and an inner surface 522. Likewise, lower member 530 comprises an outer surface 531 and an inner surface 532 that defines a hollow region or cavity 538. Excised tissue segment 399 is temporarily held within cavity 538, and thus, cavity 538 may be referred to herein as a “tissue capture chamber.”

Tissue excision device 500 functions substantially the same as tissue excision device 300 previously described. For instance, tissue excision device 500 has an opened position (not shown) in which lower member 530 extends from upper member 520 and tissue capture chamber 538 is open to the environment external tissue excision device 500; and further, tissue excision device 500 has a closed position (FIGS. 21 and 22) in which tissue capture chamber 438 is closed off by upper member 420. However, tissue excision device 500 further comprises an embodiment of a tissue retrieval device 560 slidingly disposed within cavity or tissue capture chamber 538.

Referring still to FIGS. 21 and 22, tissue retrieval device 560 comprises an elongate body 565 that includes at least one barb 561 at its distal end. Body 561 may comprise a rod, wire, or other suitable elongate member that may be inserted into and withdrawn from tissue capture chamber 538 in direction of arrows 590, 591 substantially parallel to axis 550. In addition, body 561 is preferably sufficiently rigid so that it does not bend or fold upon itself when it is inserted into and advanced within tissue capture chamber 538 and excised tissue segment 399.

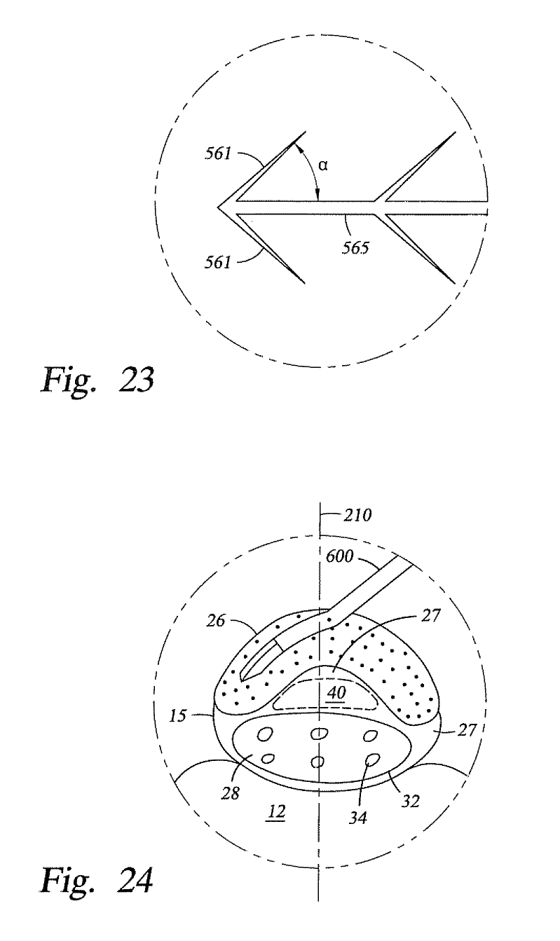

In the embodiment shown in FIGS. 21 and 22, tissue retrieval device 560 includes a plurality of barbs 561 at its distal end. Barbs 561 are generally extensions or projections from body 565. Barbs 561 are preferably configured such that they may advance into excised tissue segment 399 in the direction of arrow 591, but grasp and pull excised tissue segment 399 when retracted in the direction of arrow 590. Thus, barbs 561 are preferably angled back at an angle α relative to body 565 as best seen in FIG. 23. Angle α between each barb 561 and body 561 is preferably less than 90°, and more preferably between 15° and 75°.

Referring again to FIGS. 21 and 22, the distal portion of tissue excision device 500 is shown following the excision of tissue segment 399 from surrounding tissue 398. Further, in this view, tissue excision device 500 has not been withdrawn from tissue 398 or the patient, but rather remains within tissue 398. Without the need to withdraw tissue excision device 500, excised tissue segment 399 may be retrieved and removed from tissue excision device 500 by tissue retrieval device 560. Specifically, tissue retrieval device 560 is advanced within tissue capture chamber 538 in the direction of arrow 591 towards excised tissue segment 399 (FIG. 21). The distal end of tissue retrieval device 560, including barbs 561, contact excised tissue segment 399 and are urged into excised tissue segment 399 in the direction of arrow 591. Once one or more barbs 561 are completely disposed within excised tissue segment 399, tissue retrieval device 560 may be retracted and withdrawn from tissue capture chamber 538 in the direction of arrow 590. As tissue retrieval device 560 is withdrawn, barbs 561 engage and grasp excised tissue segment 399 and pull excised tissue segment 399 along with tissue retrieval device 560. Tissue retrieval device 500 along with the retrieved excised tissue segment 399 are then completely withdrawn and removed from tissue excision device 500. Excised tissue segment 399 may then be removed from barbs 561 and the process repeated to retrieve additional excised tissue from tissue capture chamber 538. In this manner, excised tissue segment 399 may be retrieved and removed from tissue excision device 500 without withdrawal, removal, or repositioning of tissue excision device 500. It should be appreciated that the positioning and movement of tissue retrieval device 560 may be controlled by manipulating body 565 external to the patient and tissue excision device 500.

Although tissue retrieval device 560 illustrated in FIGS. 21-23 employs one or more barbs to grasp excised tissue segment 399, alternative suitable devices and methods may also be employed to retrieve and remove excised tissue segment 399 from a tissue excision device (e.g., tissue excision device 300, 400, 500) without the need to withdraw, remove, or reposition the tissue excision device. Other suitable devices and methods for retrieving excised tissue segment 399 from embodiments of the tissue excision device described herein are disclosed in U.S. application Ser. No. 11/555,899 filed concurrently herewith, which is hereby incorporated herein by reference in its entirety.

The embodiments of the tissue excision devices disclosed above (e.g., tissue excision device 300, 400, 500) have been illustrated and described as substantially elongate, straight devices. However, it should be appreciated that different embodiments of the tissue excision devices may include a distal portion that is curved or contoured to facilitate insertion into a target tissue. For instance, referring briefly to FIG. 24, the distal portion of tissue excision device 600 may be non-linear or contoured to fit within the ligamentum flavum 26. The contour may be such that tissue excision device 600 fits easily between the spinal lamina and safety zone 40. Similarly, the distal portion of tissue excision device 600 may be shaped or contoured to fit into any other cavity or region of the body.

The components of the tissue excision devices described herein may comprise any suitable material(s) including, without limitation, metals (e.g., stainless steel, titanium, etc.), non-metals (e.g., polymer, composites, etc.) or combinations thereof. The components are preferably manufactured from durable biocompatible materials such as 400 series stainless steel, 17 series stainless steel, 300 series stainless steel, or titanium. The body of the tissue excision devices described herein, including the upper member and the lower member, as well as the tissue ejectors and retrieval devices described herein each preferably comprise a sufficiently rigid material(s) capable of maintaining its shape and configuration when inserted into and advanced through tissue.

In addition, the components of the tissue excision devices described herein may be fabricated by any suitable method(s) including, without limitation, casting or molding, machining, laser-cutting, electromechanical deposition (EMD), electro-polishing, or combinations thereof. In some embodiments, cutting edges or tips (e.g., cutting tips 325, 335) may be electro polished to enhance sharpening. Further, the components may be assembled by any suitable method including without limitation welding, press fitting, or combinations thereof. In some embodiments, the inner surface of the lower member (e.g., inner surface 332 of lower member 330) defining the tissue capture chamber (e.g., tissue capture chamber 338) may be textured or roughened to enhance gripping of the excised tissue segment within cavity 338 as the tissue excision device is closed. Such texturing may be achieved by diamond knurling, sand blasting, bead blasting, plasma etching, media blasting, or any combination thereof.

In the manner described, embodiments of the tissue excision devices disclosed herein (e.g., tissue excision device 300, 400, 500, 600, etc.) may be employed to excise and remove segments of a tissue of interest. For instance, the embodiments described herein may be used to excised portions of an enlarged ligamentum flavum, thereby decompressing the stenosed ligamentum flavum and relieving the symptoms associated therewith. The process of inserting the tissue excision device into the tissue of interest (e.g., tissue 398) in the opened position, allowing a portion of the tissue of interest to slide within the tissue capture chamber, closing the tissue excision device to excise the segment of tissue and capture it within the tissue capture chamber, and removal of the excised tissue segment from the tissue capture chamber may be repeated until the desired amount of tissue has been excised and removed.

Embodiments of tissue excision tools, devices, and methods disclosed herein may take several forms and may be used according to the ILAMP method described above, or used according to alternative MILD procedures (e.g., MILD procedure schematically illustrated in FIG. 5). One such alternative MILD procedure is disclosed in U.S. application Ser. No. 11/193,581, which is hereby incorporated herein by reference in its entirety.

In addition, the methods and procedures disclosed herein may be facilitated by a kit for performing a spinal procedure (e.g., percutaneous decompression of enlarged ligamentum flavum). Such a kit preferably includes the basic components employed in one or more of the methods disclosed herein. For instance, in one embodiment, the kit preferably includes an insertion member (e.g., cannula) for accessing the epidural space, a contrast medium to create a safety zone, a tissue excision device (e.g., tissue excision device 300, 400, 500) to cut tissue segments, and a tissue retrieval device (e.g., tissue retrieval device 560) to retrieve and remove the excised tissue segment from the tissue excision device. Depending on the application fewer or more components may be included in the kit.

While preferred embodiments of this invention have been shown and described, modifications thereof can be made by one skilled in the art without departing from the scope or teaching herein. For example, the means by which the safety zone is formed may be varied, the shape and configuration of the tissue excision devices may be varied, and the steps used in carrying out the technique may be modified. Accordingly, the invention is not limited to the embodiments described herein, but is only limited by the claims that follow, the scope of which shall include all equivalents of the subject matter of the claims. Likewise, the sequential recitation of steps in a claim, unless explicitly so stated, is not intended to require that the steps be performed in any particular order or that a particular step be completed before commencement of another step.

What is claimed is:

1. A device for percutaneously excising tissue comprising:
an elongate body including a first member having a distal cutting end and a second member that slidingly engages the first member, wherein the second member includes a tissue capture chamber having an opening facing the first member; and
wherein the first member is moveable relative to the second member between an opened position and a closed position, wherein the first member is disposed across the tissue capture chamber of the second member when the first member is in the closed position.

2. The tissue excision device of claim 1 wherein the first member and the second member move substantially parallel to each other.

3. The tissue excision device of claim 1 wherein the body has a longitudinal axis and the first member moves substantially parallel to the longitudinal axis relative to the second member.

4. The tissue excision device of claim 1 wherein the second member comprises a distal end, wherein the distal cutting end of the first member is adjacent the distal end of the second member when the first member is in the closed position.

5. The tissue excision device of claim 4 wherein the distal end of the second member is a distal cutting end.

6. The tissue excision device of claim 1 wherein the first member has an inner surface facing the second member and an outer surface facing away from the second member and wherein the second member includes an inner surface defining the tissue capture chamber, an outer surface facing away from the first member, and a dynamic sliding surface extending between the inner surface and outer surface of the second member, the dynamic sliding surface slidingly engaging the inner surface of the first member.

7. The tissue excision device of claim 3 wherein the outer surface of the first member is disposed substantially at a radius R₁ and the outer surface of the second member is disposed at a radius R₂, wherein R₁ is substantially the same as R₂.

8. The tissue excision device of claim 7 wherein the inner surface of the second member that defines the tissue cavity chamber is located substantially at a radius R₃ that is less than radius R₂.

9. The tissue excision device of claim 7 further comprising a sleeve having an inner surface defining a through bore, wherein the body is at least partially disposed within the through bore.

10. The tissue excision device of claim 9 wherein the outer surface of the first member and the outer surface of the second member slidingly engage the inner surface of the sleeve.

11. The tissue excision device of claim 5 wherein the distal cutting end of the first member comprises a beveled surface extending between and the inner surface and the outer surface of the first member that forms a sharpened cutting tip.

12. The tissue excision device of claim 6 wherein the dynamic sliding surface of the second member comprises a recess that mates with a projection extending from the first member.

13. The tissue excision device of claim 3 further comprising a rail system between the first member and the second member, wherein the rail system restricts the movement of the first member relative to the second member to directions substantially parallel to the axis of the body.

14. The tissue excision device of claim 13 wherein the tissue retrieval device comprises an elongate body having a distal end including at least one barb.

15. The tissue excision device of claim 1, wherein at least a portion of the body is contoured.

16. The tissue excision device of claim 6 further comprising:
a tissue ejector including a plunger coupled to an ejector shaft, wherein the tissue ejector is slidingly received within the tissue capture chamber of the second member; and
wherein the plunger slidingly engages the inner surface of the second member.

17. A method for treating stenosis in a spine of a patient having a median plane, the spine including a spinal canal having a posterior surface, a dural sac and an epidural space between the posterior surface and dural sac, the location of the stenosis determining a region of interest in the spine, comprising:
a) providing a tissue excision device comprising:
a first member and a second member that slidingly engages the first member;
wherein the second member includes a cavity having an opening that faces the first member; and
wherein the first member and the second member are movable relative to one another between an opened position and a closed position, wherein the first member is disposed across the cavity of the second member when the first member is in the closed position;
b) positioning the tissue excision device adjacent the region of interest;
c) opening the cavity of the tissue excision device by sliding the first member relative to the second member;
d) inserting the tissue excision device into tissue in the region of interest; and
e) closing the cavity of the tissue excision device by sliding the first member relative to the second member; and
f) capturing an excised tissue segment within the cavity of the second member.

18. The method of claim 17 further comprising withdrawing the tissue excision device from the tissue in the region of interest.

19. The method of claim 18 further comprising opening the cavity of the tissue excision device by sliding the first member relative to the second member and emptying the excised tissue segment from the cavity of the second member with a plunger slidingly disposed within the cavity of the second member.

20. The method of claim 17 wherein the tissue excision device further comprises a tissue retrieval device slidingly disposed within the cavity of the second member.

21. The method of claim 20 further comprising retrieving and removing the excised tissue segment from the cavity of the second member with the tissue retrieval device.

22. The method of claim 17 wherein a portion of the patient's ligamentum flavum occupies the region of interest, and wherein excised tissue segment comprises a portion of the ligamentum flavum from the region interest.

23. A kit for performing a procedure on a spine, the spine including an epidural space containing a dural sac, the kit comprising:
a tissue excision device, wherein the tissue excision device comprises:
a first moveable member;
a second moveable member including a tissue capture chamber having an opening facing the first moveable member, wherein the second moveable member slidingly engages the first moveable member;
wherein the first moveable member is slidable between a first position and a second position relative to the second member;
wherein the first member is disposed across the cavity when the first moveable member is in the first position, and wherein the cavity is at least partially open when the first member is in the second position; and
a tissue retrieval device.

24. The kit of claim 23 further comprising a volume of a contrast medium adapted to be inserted into the epidural space by the insertion member and expanded so as to compress a portion of the thecal sac and provide a safety zone within the epidural space.

25. The kit of claim 24 wherein the tissue retrieval device comprises an elongate body having a distal end including at least one barb.
